(19) United States
(12) Patent Application Publication
(10) Pub. No.: US 2024/0081752 A1
(43) Pub. Date: Mar. 14, 2024

(54) Vital Signs Monitoring Device

(71) Applicant: Nordiq Products AS, Stjørdal (NO)

(72) Inventors: Mats Hetling, Trondheim (NO); Lise Lyngsnes Randeberg, Trondheim (NO); Robert Ditlefsen, Østre Gausdal (NO); Emil Absjørnslett, Trondheim (NO); Rolf Sellesbakk, Trondheim (NO); Gregoir Bonnemaire, Trondheim (NO)

(21) Appl. No.: 18/266,869

(22) PCT Filed: Dec. 21, 2021
(86) PCT No.: PCT/EP2021/087099

§ 371 Date: Jun. 13, 2023

(30) Foreign Application Priority Data
Dec. 21, 2020 (GB) 2020294.1

(51) Int. Cl. A61B 5/00; A61B 5/0205; A61B 5/1455

(52) U.S. Cl. CPC A61B 5/746; A61B 5/0205; A61B 5/14551; A61B 5/6822; A61B 5/6831; A61B 5/6833; A61B 5/7405; A61B 5/7425; A61B 5/02416; A61B 2562/0204

(57) ABSTRACT

A monitoring device configured to be removably attached to a patient's neck comprises an optical unit for monitoring one or more vital signs, and an acoustic unit for monitoring one or more vital signs.

### FIELD OF THE DISCLOSURE

The present invention relates to a monitoring device configured to be removably attached to a patient's neck, for monitoring the patient's vital signs.

### BACKGROUND

The first priority in all types of medical care, such as hospital or pre-hospital care, critical care, emergency medicine and anaesthesia, is maintenance of an open airway. Even a short-lasting loss of airway patency can be life-threatening for a patient, and so monitoring breathing and other vital signs is crucial.

Monitoring a patient's airway manually to determine whether the patient's vital signs are adequate requires substantial clinical training and awareness, and the airway must be continuously assessed to detect any drop in airway patency. Moreover, this can be challenging in stressful emergency situations, or when it is necessary to monitor multiple patients at once such as in in busy hospitals, field hospitals, mass casualty incidents, intra-hospital transportation, and military settings.

Monitoring devices can aid health care workers in monitoring an airway, but these are typically bulky or do not provide any reliable real-time direct monitoring of the airway. Instead, these devices typically trigger alarms when airway failure is detected, but this detection can often occur too late, which can lead to the patient suffering from complex conditions such as oxygen desaturation and arrhythmia.

Traditional pulse oximetry monitors (which measure on the fingertip) give a late indication of airway obstruction, and are unreliable in hypothermic, shocked, and hypotensive patients.

Capnography, which is one of the more robust monitoring methods, requires expensive sensors and is not often suitable in many situations.

There is therefore a need to provide a monitoring device that is able to detect and monitor vital signs both readily and reliably in a way that can be easily monitored by health care workers in a broad range of emergency and non-emergency situations. A device is also needed to provide an earlier, more decisive, warning of loss of airway patency. A device that is both easy to use and versatile is needed.

### SUMMARY

Viewed from a first aspect, there is provided a monitoring device configured to be removably attached to a patient's neck, the monitoring device comprising: an optical unit for monitoring one or more vital signs; and an acoustic unit for monitoring one or more vital signs.

The optical unit may comprise an optical sensor. For example, the optical sensor may be a reflectance pulse oximeter.

The vital signs measured by the optical sensor may include one or more of (and preferably all of): blood oxygen saturation, pulse rate, and respiration rate (breathing rate).

In order to measure blood oxygen saturation the optical sensor may measure reflectance at two or more wavelengths of light. A first wavelength may be one at which the light absorption is the same in oxygenated haemoglobin and deoxygenated haemoglobin, and a second wavelength may be one at which there is a substantial difference in light absorption for oxygenated and deoxygenated haemoglobin. Known algorithms may be used to determine the blood oxygen saturation based on the measurements of reflectance.

In order to measure pulse rate using the optical sensor, measurement of reflectance of light at a single wavelength may be sufficient. As the heart beats, blood pulses through the capillaries with the same frequency as the heartbeat. This pulsation effectively means that the volume of blood within the capillaries is changing. The changing volume in turn gives rise to a change in the amount of light absorbed by the blood, and hence the amount reflected. An estimation of the pulse rate may therefore be obtained by the optical sensor by measuring the time-dependent fluctuation in the reflected light. Known algorithms may be used to determine the pulse rate based on the time-dependent fluctuations in reflectance.

It is also possible to extract the respiration rate information using an optical sensor, because the respiratory activity affects the photoplethysmographic waveform which is obtained. Known algorithms may be used to determine the respiration rate based on the photoplethysmographic waveform.

The optical unit may comprise a lighting unit capable of emitting light in at least two different wavelength ranges onto the skin. The lighting unit may comprise a single light source capable of being controlled to emit light in the at least two different wavelength ranges. The light source may be an LED. Alternatively, the optical unit may comprise two light sources, each configured to emit light in a different wavelength range. The light sources may be LEDs. In one example, a first wavelength range has a peak at 805 nm and a second wavelength range has a peak at 660 nm.

The acoustic unit may comprise an acoustic sensor. For example, the acoustic sensor may be a piezoelectric sensor or a microphone. In embodiments in which the acoustic sensor is a microphone, the microphone may be a MEMS microphone. In embodiments in which the acoustic sensor is a piezoelectric sensor, the piezoelectric sensor may comprise a piezoelectric disc inside a cup with a membrane positioned towards the patient's skin.

The vital signs measured by the acoustic sensor may include one or more of (and preferably all of): the respiration rate, pulse rate and airway patency.

Airway patency is a measure of how obstructed the airway is. The monitoring device may monitor for signs of a partially obstructed or fully obstructed airway.

The sound of breathing through a partially obstructed airway is different from the sound of breathing through an open airway. As airway patency decreases, the normal whispering sound of air passing through the airway diminishes and other sounds like wheezing, rattling or snoring may be heard. Therefore, relatively loud (compared to the sound of normal breathing), repeating sounds such as snoring, wheezing and rattling may indicate that the airway may be compromised. Such sounds may be identified using, for example, a trained machine learning/deep learning model, trained on data including such sounds.

If the airway is fully obstructed, the loss of airway patency may be determined by detecting that the respiration rate has dropped to zero.

Loss of airway patency results in the pulse rate increasing initially, and then falling as the blood oxygen saturation drops. The rate of respiratory effort will also rise at first and then drop, but this does not give rise to actual respiration due to the obstructed airway. It only results in diaphragm contraction and negative pressure inside the chest. Loss of airway patency eventually leads to cardiac arrest. This could happen as early as 2 minutes or as late as 10 minutes after breathing ceases, or even later in hypothermia and depending on the patient age, situation and so on. By detecting loss of airway patency, the monitoring device ensures that the patient receives medical care before the patient's condition deteriorates.

Respiration rate and pulse rate may respectively be measured by the acoustic sensor simply by listening for the sounds associated with breathing in and out, and for the sound of the pulse. Known algorithms may be used to determine the respiration rate and pulse rate from the measured sounds.

The use of two different sensor types within the monitoring device helps to improve the reliability of the measured vital signs. Each of the sensors can act as a back-up or check for the values measured by the other sensor. For example, if the values for the same vital sign measured by each sensor differ, an alert can be issued by the monitoring device indicating that there may be a fault with one of the sensors. For example, the measurements of the respiration rate and pulse rate from the acoustic sensor may be used as a check against the respiration rate and pulse rate measured by the optical sensor.

By placing the monitoring device on the neck, more accurate and timely measurements of respiration rate, pulse rate, blood oxygen saturation and airway patency can be achieved without the need for bulky monitoring devices.

The acoustic unit may comprise an environmental noise sensor configured to measure unwanted environmental noise (i.e. noise that is unrelated to the patient's vital signs) in order that such unwanted environmental noise can be subtracted from the signals measured by the first acoustic sensor. The environmental noise sensor may for example be a microphone.

The acoustic unit may comprise a data acquisition module, which includes the acoustic sensor and is configured to amplify the sounds detected by the acoustic sensor and convert them to a digital signal.

Similarly, the optical unit may comprise a data acquisition module, which includes the optical sensor.

The monitoring device may comprise a signal-processing module which may receive the digital signals from the data acquisition modules of the optical unit and the acoustic unit.

The signal-processing module may receive the digital signals from the data acquisition modules of the optical unit and the acoustic unit, and may be configured to filter the digital signals and remove noise from the digital signals.

The monitoring device may comprise a data analysis module configured to determine the respiration rate, pulse rate, blood oxygen saturation, and airway patency. Optionally, the data analysis module determines the respiration rate, pulse rate, blood oxygen saturation, and airway patency based on data output from the signal-processing module.

The monitoring device may comprise a single processor configured to carry out the processes carried out by the data acquisition modules of the acoustic and optical units, the signal-processing module and the data analysis module.

In the foregoing, an embodiment is described in which the monitoring device comprises a single processor which receives data streams from both the optical sensor and the acoustic sensor. In an alternative embodiment, the monitoring device comprises two processors, each of which is dedicated to analysing a single data stream; a first processor is dedicated to analysing data from the optical unit, and a second processor is dedicated to analysing data from the acoustic unit. Each processor then comprises a data acquisition module, signal processing module, and data analysis module.

The monitoring device may be configured to self-calibrate once placed on the patient's neck and activated. For example, the monitoring device may compare the sensor signals against internal or external references.

The monitoring device may monitor the one or more vital signs by taking measurements at a pre-determined sampling rate. The sampling rate may be the same for the acoustic sensor and the optical sensor, or the sampling rate for each may differ. The pre-determined sampling rate may be in the range of 5 to 50 measurements per second, for example 10 to 30 measurements per second, and optionally is approximately 20 measurements per second.

The monitoring device may recalculate the value for the one or more vital signs using data from a predetermined measurement time interval (for example, 10 to 20 seconds), and may recalculate the value on a rolling basis at predetermined intervals (for example, 1 to 5 seconds). These values may be the same, or may differ, for each vital sign.

For example, the monitoring device may recalculate the value of respiration rate every 5 seconds, based on data acquired in the preceding 20 seconds. The pulse rate has a higher frequency than respiration rate, and so the pulse rate may be updated more frequently and based on data taken over a lesser amount of time, compared to the respiration rate. So, for example, the monitoring device may recalculate the value of pulse rate every 1 second, based on data acquired in the preceding 10 seconds.

As noted, above, in order to measure blood oxygen saturation the optical sensor may measure reflectance at two or more wavelengths of light. Measurement of blood oxygen saturation therefore may involve applying a sequence of light at different wavelengths and measuring the reflectance during that sequence, and then calculating an updated value of blood oxygen saturation at the end of each sequence.

The monitoring device may comprise a display for displaying the measurements of the one or more vital signs. As will be appreciated from the foregoing, the display is integrated into the monitoring device, and so is present on the device attached to the patient's neck.

The monitoring device may be configured to monitor the vital signs for an initial period of time before any measurements of vital signs are displayed on the device. Advantageously, this ensures that an accurate measurement of the vital signs is obtained, before displaying the measurements.

The monitoring device may update the displayed measured value for the one or more vital signs at predetermined display intervals. The pre-determined display interval may be in the range of 5 to 30 seconds, for example 5 to 20 seconds.

The provision of the monitoring device and the display for displaying the results on the neck means that the health care worker can easily view the patient's vital signs without having to reassess their airway manually at regular intervals. The health care worker will therefore be able to see whether the patient's vital signs are at an acceptable level, or if they are not, in which case the patient may require immediate attention.

The monitoring device is particularly useful in situations where multiple patients need to be monitored simultaneously, with each patient being provided with their own monitoring device.

The monitoring device may be removably attached to the neck of the patient using an adhesive pad. Accordingly, this allows the monitoring device to be attached to the patient in a non-invasive manner. Moreover, the adhesive pad makes mounting of the monitoring device easy to do and requires no clinical training. This is preferable to complex invasive procedures for monitoring the airway which require substantial equipment.

The monitoring device may be removed by pulling the device off the neck of the patient. The adhesive pad may then be removed and replaced with a new adhesive pad when required.

The monitoring device may be mounted to the neck of the patient using a strap or band. The strap or band may be non-strangulating and may also be simple and easy to attach. This is beneficial in situations where the adhesive pad is not sufficient or secure enough, for example if the patient is being transported or a procedure is being carried out on the patient that may result in them being moved such that the monitoring device may fall off.

The non-strangulating strap or band may be sized such that it does not encircle the entire circumference of the neck.

The simplicity of the monitoring device enables it to be mounted by an untrained person to monitor vital signs of a patient. This is of particular benefit for home use and is advantageous over capnography-based systems which require lots of equipment. Moreover, the monitoring device is small in size and low in weight (which is important given that it is located on the neck), meaning that it is portable (i.e. can be readily carried in a person's hands, without assistance) and easily stored. The monitoring device can also be operated using one hand only.

The monitoring device may be set with pre-defined acceptable ranges for each of the one or more vital signs (i.e. comprising an upper and lower limit which the vital signs should fall within). Alternatively, the acceptable ranges may be set by a user with a certain high-level of authorisation, (i.e. not necessarily possessed by the health care worker) depending on the patient and/or the environment. This allows the acceptable ranges to be modified if certain patients have vital signs which are normally outside of the expected range. Alternatively, in some environments the expected values may differ, and/or the health care worker may be working under excessive time pressure (for example, they may be attending to multiple patients under emergency conditions) such that they can only attend to a patient once the vital signs reach a certain point which in other situations would have already required attention.

If the one or more vital signs goes outside the acceptable range, a timer may be started. If the vital sign does not return to within the acceptable range within a pre-set time limit, the monitoring device may issue an alert. This provides an immediate warning to the health care worker that a patient's airway may be blocked and that they require immediate attention. Knowing that any significant change in the vital signs will cause an alert, the health care worker does not need to constantly monitor the patient or manually assess their airway frequently. This is especially useful if a single care worker is monitoring multiple patients simultaneously. Early detection of a loss of airway patency can greatly improve the chances of the patient surviving.

Where two values of the same vital sign are obtained by the monitoring device (for example, pulse rate and respiration rate, which each may be measured by the acoustic sensor and optical sensor), in one embodiment, the values for a given vital sign from both the acoustic sensor and optical sensor are required to go out of range before a timer starts. Alternatively, the timer may be started by only one of the values from either the acoustic sensor or optical sensor going out of the range.

The monitoring device may be set with the time limit, but also the time limits may be set by a user with a certain high-level of authorisation, (i.e. not necessarily possessed by the health care worker) (i.e. the time limits may be customisable) depending on the patient and/or the environment.

The acceptable range for blood oxygen saturation may be 90% to 100%, for example. If the blood oxygen saturation goes outside of this range, a timer may be started, and if the blood oxygen saturation does not return to the acceptable range within a pre-set time limit (for example, 15 seconds), an alert may be issued.

The acceptable range for pulse rate may be 50 to 120 beats per minute, for example. If the blood pulse rate goes outside of this range, a timer may be started, and if the pulse rate does not return to the acceptable range within a pre-set time limit (for example, 15 seconds), an alert may be issued.

The acceptable range for respiration rate may be 8 to 27 breaths per minute, for example. If the respiration rate goes outside of this range, a timer may be started, and if the respiration rate does not return to the acceptable range within a pre-set time limit (for example, 15 seconds), an alert may be issued.

If the vital sign returns to within the acceptable range within the pre-set time limit, the timer may be re-set to zero. Monitoring of the vital signs may then continue as normal.

The monitoring device may be configured to monitor the rate of change of the measurements of the vital signs, for example, their first derivative with respect to time. An alert may be issued if the rate of change of the measurements of a vital sign is outside of a predetermined acceptable range or exceeds a predetermined limit for a respective pre-set period of time. For pulse rate and respiration rate, the predetermined range may range from a negative value of rate of change, to a positive value of the rate of change, i.e. both a decreasing value or an increasing value of the vital sign, both outside of the predetermined range, would trigger the alert. For the blood oxygen saturation, only a decrease may be a concern, so an alert may be issued for any negative rate of change which has an absolute value (a modulus) greater than a predetermined limit for longer than a pre-set period of time.

Where two values of the same vital sign are obtained by the monitoring device (for example, pulse rate and respiration rate, which each may be measured by the acoustic sensor and optical sensor), in one embodiment, the values for rate of change of the measurements of the vital sign from both the acoustic sensor and optical sensor are required to go out of range before a timer starts. Alternatively, the timer may be started by only one of the values for rate of change of the measurements of the vital sign from either the acoustic sensor or optical sensor going out of the range.

A timer may begin when the rate of change of the measurements of a vital sign is outside of a predetermined acceptable range or exceeds a predetermined limit.

For example, if a patient's respiratory rate changes by more than ±30% (and optionally remains outside of this range for a 10 minute period), an alert may be issued.

For example, if a patient's pulse rate changes by more than ±40% (and optionally remains outside of this range for a 10 minute period), an alert may be issued.

For example, if a patient's blood oxygen decreases by more than 5% (and optionally remains outside of this limit for a 10 minute period), an alert may be issued.

If the rate of change of the measurements of the vital sign returns to within the predetermined acceptable range/limit within the pre-set time limit, the timer may be re-set to zero. Monitoring of the vital signs may then continue as normal.

The monitoring device may be set with the predetermined acceptable range and predetermined limit on the rate of change of the measurements of a vital sign, as well as the period of time, but also these may be set by a user with a certain high-level of authorisation, (i.e. not necessarily possessed by the health care worker) depending on the patient and/or the environment.

An alert may be issued in the foregoing cases, even if the value for the one or more vital signs is within the predetermined acceptable range for said value.

If the one or more vital signs (for example, respiration rate and/or pulse rate) recorded by the optical sensor and the acoustic sensor diverge by more than a pre-set threshold value, a timer may be started. If the divergence between the vital signs recorded by the optical sensor and the acoustic sensor does not drop back to below the threshold value within a pre-set time limit, the monitoring device may issue an alert. This is beneficial because it can indicate to the health care worker that there is a fault with one of the sensors. The monitoring device may be set with the threshold value for how much the one or more vital signs from each sensor may diverge. As with the pre-defined acceptable ranges for the vital signs, the threshold values for divergence and the time limits are stored in the monitoring device. The threshold values and time limits may differ depending on the vital sign.

The acceptable divergence for respiratory rate may be 3 breaths per minute, for example. If the values measured by the optical sensor and the acoustic sensor diverge by more than this, a timer may be started. If the divergence does not fall back below the threshold value within a pre-set time limit (for example, 1 minute), an alert may be issued.

The acceptable divergence for pulse rate may be 12 beats per minute, for example. If the values measured by the optical sensor and the acoustic sensor diverge by more than this, a timer may be started. If the divergence does not fall back below the threshold value within a pre-set time limit (for example, 30 seconds), an alert may be issued.

If the divergence returns to below the pre-set threshold value within the pre-set time limit, the timer may be re-set to zero. Monitoring of the vital signs may then continue as normal.

The monitoring device may also be configured to detect decreasing, or loss, of airway patency. On detection of one or more of snoring, wheezing or rattling, a timer may be started, and an alert may be issued if the snoring, wheezing or rattling continues for a predetermined time. The predetermined time may be 5 to 10 minutes, for example. The time period may be set in consideration of the fact that it is advantageous to avoid issuing alarms too frequently, particularly where they are not warranted (for example if a patient is semi-conscious, they could make noises that could potentially trigger the alarm) as this can cause the health care worker to become fatigued attending to the alarm, and may even lead to them ignoring the alarm.

If the airway is fully obstructed, the loss of airway patency may be determined by detecting that the respiration rate has dropped to zero. If the respiration rate drops to zero, a timer may be started and an alert may be issued after a pre-set time—typically around 20 seconds, for example.

The alert may be an audible alert, for example an alarm. Alternatively, the alert may be a visual alert, for example an indicator light. As a further alternative, the processor may issue both an audible and a visual alert. An audible alert is beneficial in that the health care worker is more likely to notice it than a visual alert. However, audible alerts may not be preferable in certain environments such as military situations. In this instance, the audible alert may be deactivated and the health care worker can rely solely on a visual alert, for example an indicator light.

If the monitoring device is low on power, the monitoring device may issue an alert for maintenance. This alert for maintenance may be using the audible alert and/or the visual alert.

The alerts can also be used to notify the health care worker that the device is operating correctly. For example, the monitoring device may issue alerts at regular intervals.

The history of the one or more vital signs and/or alerts may be stored in an integrated storage means in the monitoring device. This can allow the health care worker to monitor the patient's vital signs over a prolonged period and monitor when alerts have occurred and the speed with which they were attended to.

The monitoring device may further comprise a wireless interface (for example a Bluetooth interface) for communication with an external device. The external device may be a mobile phone, laptop, desktop computer or tablet, for example. The external device can be used to customise the pre-defined ranges and thresholds discussed above (for example by a user with a high level of authorisation, not necessarily possessed by the health care workers). A single external device may be able to communicate with multiple monitoring devices which is useful if multiple patients are being monitored simultaneously. The wireless interface may also be used to transmit the history of the one or more vital signs and/or alerts to the external device.

The monitoring device may also transmit the history of the one or more vital signs and/or alerts to a remote storage sever via Wi-Fi or a 3G/4G/5G network. The remote storage server can receive data from multiple monitoring devices simultaneously. This allows for a complete database to be formed from numerous monitoring devices and allows for remote monitoring from a central location. The remote storage server may be monitored at a central location, for example, in the case of a field hospital or military operations, it may be at the location of the nearest doctor or nurse who is not able to be on hand with all the patients simultaneously.

Rather than the monitoring device transmitting the history of the one or more vital signs and/or alerts to a remote storage sever directly, the monitoring device may transmit the data to the external device, which may relay the data to the remote storage server.

Viewed from a second aspect, there is provided a method of monitoring one or more vital signs of a patient using a monitoring device, the monitoring device comprising an optical unit for monitoring one or more vital signs, and an acoustic unit for monitoring one or more vital signs, wherein the method comprises: attaching the monitoring device to a patient's neck; and monitoring the one or more vital signs.

This method allows the patient's airway patency, respiration rate, pulse rate and blood oxygen saturation to be monitored without the need for constant reassessment by a health care worker.

The one or more vital signs may be monitored by taking measurements at a pre-determined sampling rate. The sampling rate may be the same for the acoustic unit and the optical unit, or the sampling rate for each may differ. The pre-determined sampling rate may be in the range of 5 to 50 measurements per second, for example 10 to 30 measurements per second, and optionally is approximately 20 measurements per second.

The method may comprise recalculating the value for the one or more vital signs using data from a predetermined measurement time interval (for example, 10 to 20 seconds), on a rolling basis at predetermined intervals (for example, 1 to 5 seconds). These values may be the same, or may differ, depending on the vital sign.

For example, the method may recalculate the value of respiration rate every 5 seconds, based on data acquired in the preceding 20 seconds. The pulse rate has a higher frequency than respiration rate, and so the pulse rate may be updated more frequently and based on data taken over a lesser amount of time, compared to the respiration rate. So, for example, the method may recalculate the value of pulse rate every 1 second, based on data acquired in the preceding 10 seconds.

As noted, above, in order to measure blood oxygen saturation the optical sensor may measure reflectance at two or more wavelengths of light. Measurement of blood oxygen saturation therefore may involve applying a sequence of light at different wavelengths and measuring the reflectance during that sequence, and then calculating an updated value of blood oxygen saturation at the end of each sequence.

The method may comprise monitoring the vital signs for an initial period of time before any measurements of vital signs are displayed on the device. Advantageously, this ensures that an accurate measurement of the vital signs is obtained, before displaying the measurements.

The method may comprise determining whether the one or more vital signs falls within or outside pre-defined acceptable ranges.

If the one or more vital signs are outside of the respective pre-defined range a timer may be started. If the vital sign does not return to within the acceptable range within a pre-set time limit, the method may comprise issuing an alert, wherein the alert may be an audible alert and/or a visual alert. This alerts the health care worker as soon as the patient exhibits signs of a loss of airway patency and allows the patient to receive immediate attention. The use of an audible alert means that the health care worker is not required to monitor the monitoring device directly, they are merely required to be in the vicinity such that they can hear the audible alert. The audible alert may be an alarm.

The visual alert is beneficial in situations where an audible alert is not suitable, such as military environments.

The acceptable range for blood oxygen saturation may be 90% to 100%, for example. If the blood oxygen saturation goes outside of this range, a timer may be started, and if the blood oxygen saturation does not return to the acceptable range within a pre-set time limit (for example, 15 seconds), an alert may be issued.

The acceptable range for pulse rate may be 50 to 120 beats per minute, for example. If the pulse rate goes outside of this range, a timer may be started, and if the pulse rate does not return to the acceptable range within a pre-set time limit (for example, 15 seconds), an alert may be issued.

The acceptable range for respiration rate may be 8 to 27 breaths per minute, for example. If the respiration rate goes outside of this range, a timer may be started, and if the respiration rate does not return to the acceptable range within a pre-set time limit (for example, 15 seconds), an alert may be issued.

The method may further comprise monitoring the divergence in the one or more vital signs (for example, respiration rate and/or pulse rate) measured by each of the acoustic sensor and the optical sensor.

If the one or more vital signs monitored by each of the acoustic sensor and optical sensor diverge by more than a threshold value, a timer may be started. If the divergence between the vital signs recorded by the optical sensor and the acoustic sensor does not drop back to below the threshold value within a pre-set time limit, the method may further comprise issuing an alert. As above, the alert may be an audible alert or a visual alert.

The acceptable divergence for respiratory rate may be 3 breaths per minute, for example. If the values measured by the optical sensor and the acoustic sensor diverge by more than this, a timer may be started. If the divergence does not fall back below the threshold value within a pre-set time limit (for example, 1 minute), an alert may be issued.

The acceptable divergence for respiratory rate may be 3 breaths per minute, for example. If the values measured by the optical sensor and the acoustic sensor diverge by more than this, a timer may be started. If the divergence does not fall back below the threshold value within a pre-set time limit (for example, 1 minute), an alert may be issued.

The acceptable divergence for pulse rate may be 12 beats per minute, for example. If the values measured by the optical sensor and the acoustic sensor diverge by more than this, a timer may be started. If the divergence does not fall back below the threshold value within a pre-set time limit (for example, 30 seconds), an alert may be issued.

This system means that the health care worker will be notified if there is fault in one of the sensors, but small errors or discrepancies can be ignored without issuing an alert.

The use of two different sensors improves the reliability of the method for monitoring the one or more vital signs as an error in one of the sensors will be detected if it diverges significantly from the value measured by the other sensors. If only a single sensor was used, it would not be possible to determine whether the sensor was displaying the correct result without manually assessing the airway.

The method may comprise monitoring the rate of change of the measurements of the vital signs, for example their first derivative with respect to time. The method may comprise issuing an alert if the rate of change of the measurement of a vital sign with respect to time is outside of a pre-determined acceptable range (including positive and negative values of the rate of change) or exceeds a predetermined limit for a respective pre-set period of time. For pulse rate and respiration rate, the predetermined range may range from a negative value of rate of change, to a positive value of the rate of change, i.e. both a decreasing value or an increasing value of the vital sign, both outside of the predetermined range, would trigger the alert. For the blood oxygen saturation, only a decrease may be a concern, so an alert may be issued for any negative rate of change which has an absolute value (a modulus) greater than a predetermined limit for longer than a pre-set period of time.

A timer may begin when the rate of change of the measurements of a vital sign is outside of a predetermined acceptable range or exceeds a predetermined limit.

For example, if a patient's respiratory rate changes by more than ±30% (and optionally remains outside of this range for a 10 minute period), an alert may be issued.

For example, if a patient's pulse rate changes by more than ±40% (and optionally remains outside of this range for a 10 minute period), an alert may be issued.

For example, if a patient's blood oxygen decreases by more than 5% (and optionally remains outside of this limit for a 10 minute period), an alert may be issued.

The method may comprise monitoring for decreasing, or loss, of airway patency. On detection of one or more of snoring, wheezing or rattling, a timer may be started, and an alert may be issued if the snoring, wheezing or rattling continues for a predetermined time.

If the airway is fully obstructed, the loss of airway patency may be determined by detecting that the respiration rate has dropped to zero. If the respiration rate drops to zero, a timer may be started and an alert may be issued after a pre-set time—typically around 20 seconds, for example.

The method may comprise setting one or more customised acceptable ranges for the one or more vital signs, rate of change of vital signs, divergence of vital signs measured by both of the two sensors, or the respective time period for each of these. These may only be set by users with a certain high level of authorisation, and for example may not be set by the health care workers themselves.

The method according the second aspect may further comprise any of the features associated with the first aspect.

### BRIEF DESCRIPTION OF THE DRAWINGS

Certain preferred embodiments will now be described in greater detail by way of example only and with reference to the accompanying drawings in which:

FIG. 1 is a schematic view of a monitoring device comprising two sensors;

FIG. 2 is a perspective view of a monitoring device including the display;

FIG. 3 shows a monitoring device mounted to a patient;

FIG. 4A shows an adhesive pad for mounting the monitoring device;

FIG. 4B shows an adhesive pad being applied to the rear of a monitoring device;

FIG. 5 shows a schematic view of a monitoring device comprising two sensors interacting with a remote server; and

FIG. 6 shows a method of using the monitoring device.

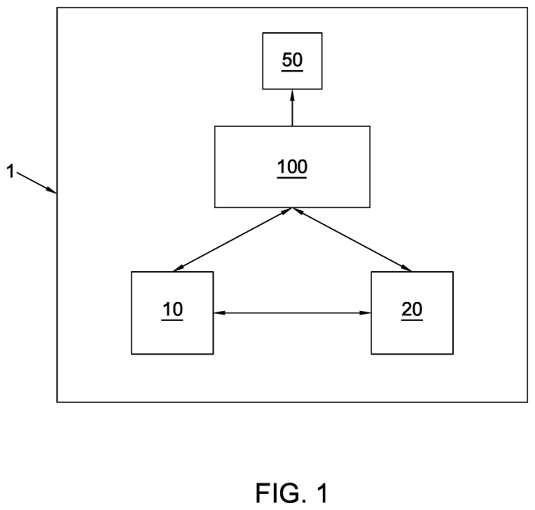

FIG. 1 shows a monitoring device 1 which is used for monitoring a patient's vital signs, and which continuously monitors the patient's respiration rate, pulse rate, blood oxygen saturation and airway patency.

### DETAILED DESCRIPTION

The monitoring device comprises a first sensor 10 and a second sensor 20, which both interact with a processor 100. The processor 100 is able to interpret the readings from the first sensor 10 and the second sensor 20 and store the results in a storage means 50. The storage means 50 also stores pre-defined ranges for each vital sign measurement, the acceptable ranges/limits for the rate of change of vital sign measurements, and the amount by which measurements of the same vital sign from the two different sensors may diverge, as well as the time periods applicable to each of these.

The first sensor 10 is an optical sensor, which in this case is a reflectance pulse oximeter sensor.

As known in the art, the reflectance pulse oximeter 10 works on the principle that oxygen-rich blood absorbs light with a different wavelength than blood which is low in oxygen, and so oxygen saturation in the blood can be measured optically. In order to measure blood oxygen saturation the optical sensor measures reflectance at two or more wavelengths of light. A first wavelength is one at which the light absorption is the same in oxygenated haemoglobin and deoxygenated haemoglobin, and a second wavelength is one at which where there is a substantial difference in light absorption for oxygenated and deoxygenated haemoglobin.

A reflectance pulse oximeter 10 can also be used to measure pulse rate. In order to measure pulse rate, measurement of reflectance of light at a single wavelength is sufficient. As the heart beats, blood will pulse through the arteries with the same frequency as the heartbeat. This pulsation effectively means that the volume of blood within the artery is changing. The changing volume in turn gives rise to a change in the amount of light absorbed by the blood, and hence the amount reflected. An estimation of the pulse rate can therefore be obtained by measuring the time-dependent fluctuation in the reflected light.

It is also possible to extract the respiration rate information from a reflectance pulse oximeter, because the respiratory activity affects the photoplethysmographic waveform which is obtained by a reflectance pulse oximeter. Small changes in this waveform can be analysed by the processor 100 and using machine learning algorithms the changes can be used to determine respiration rate. It will be appreciated that various other types of signal processing means can be used to determine the respiration rate. For example, advanced filtering techniques, transforms allowing for decomposition of the signal as well as neural networks. Further details are disclosed in “Extracting breathing rate information from a wearable reflectance pulse oximeter sensor”, W. S. Johnston, Y. Mendelson, Department of Biomedical Engineering and the Bioengineering Institute, Worcester Polytechnic Institute, September 2004.

To enable the reflectance pulse oximeter to take the necessary measurements, the monitoring device comprises two LEDs (not shown) each configured to emit light in a different wavelength range. A first LED emits light with a wavelength peak at 805 nm and a second LED emits light with a wavelength peak at 660 nm.

The second sensor 20 comprises an acoustic sensor comprising a microphone or a piezoelectric sensor. The acoustic sensor 20 can monitor the airway and provide a value of respiration rate and pulse rate which can be used as a second estimate to be compared with the same measurements from the optical sensor 10. The acoustic sensor 20 also provides an assessment of the airway patency, including whether it is healthy and unobstructed or if it is partially blocked or completely obstructed.

The sound of breathing through a partially obstructed airway is different from the sound of breathing through an open airway. As airway patency decreases, the normal whispering sound of air passing through the airway diminishes and other sounds like wheezing, rattling or snoring may be heard. Therefore, relatively loud (compared to the sound of normal breathing), repeating sounds such as snoring, wheezing and rattling may indicate that the airway may be compromised.

If the airway is fully obstructed, the loss of airway patency is indicated by detecting that the respiration rate has dropped to zero.

The processor 100 comprises a data acquisition module, a signal-processing module and a data analysis module.

The data acquisition module receives signals from the optical sensor 10 and the acoustic sensor 20.

The signal-processing module then filters the digital signal and removes noise. The processed signals are then forwarded to the data analysis module.

The data analysis module of the processor 100 is able to analyse the measurements from each sensor and provide values for respiration rate and pulse rate for both the first and second sensors 10, 20 as well as the blood oxygen saturation from the optical sensor 10 and the airway patency from the acoustic sensor 20.

The processor 100 can then cause a display 5 of the monitoring device 1 to display the data for the care provider to view and it is also able to store the measured data in the integrated storage means 50 within the monitoring device 1.

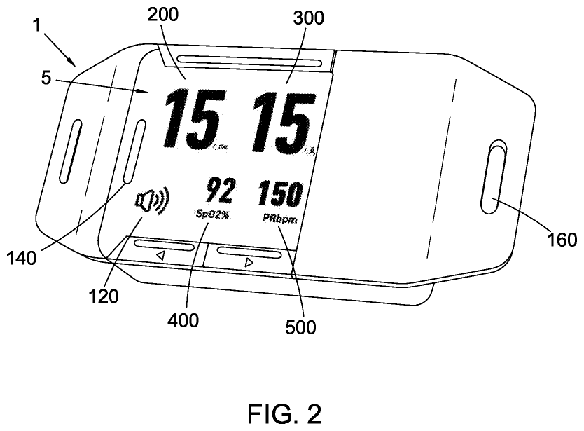

FIGS. 2 to 4 show perspective views of the monitoring device 1. As shown in FIG. 2, the monitoring device 1 comprises a display 5 for displaying the readings from the first sensor 10 and the second sensor 20. The display 5 shows the respiration rate as measured by both the first sensor 10 and the second sensor 20.

The acoustic sensor measurement 200 of the respiration rate is determined by the processor 100 using the acoustic sensor 20 as discussed above. On the display, the optical sensor measurement 300 of the respiration rate is provided adjacent to the acoustic measurement sensor reading 200 and is determined by the processor 100 using the optical sensor 10.

The display 5 also provides a measurement for the blood oxygen saturation 400 which is determined by the processor 100 using the optical sensor 10 as discussed above. The display 5 is also capable of displaying the pulse rate 500 which can be determined by the processor 100 using both the optical sensor 10 and the acoustic sensor 20. Typically, the optical sensor 10 will provide the primary reading for the pulse rate 500, and the acoustic sensor 20 will provide a secondary estimation (and a cross-check, in the event that there is a problem with the optical sensor 10).

The monitoring device 1 comprises an audible alarm 120 and a visual indicator light 140 for alerting the care provider. The audible alarm 120 and visual indicator light 140 can be used for various different alerts, such as indicating that the device has calibrated, that the patient's vital signs are outside of a pre-defined range of acceptable values, that the rate of change of a vital sign is outside of a predetermined acceptable range, or that the measurements from each sensor have diverged a significant amount (where the divergence exceeds a pre-defined threshold value). The audible alarm 120 can be deactivated in certain situations, e.g. military environments.

If any alerts are triggered, the processor stores an indication of which alert has been triggered, and the time, in the integrated storage means 50 within the monitoring device 1.

The audible alarm 120 or visual indicator light 140 also indicate when the battery of the monitoring device 1 is low.

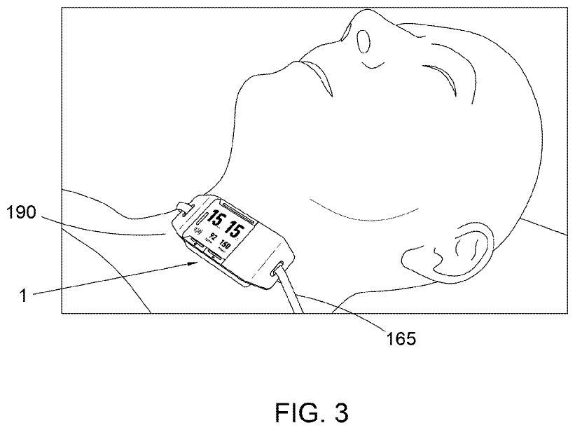

FIG. 3 shows the monitoring device 1 mounted to the patient's neck 190. The placement on the neck allows for the monitoring device 1 to be easily placed on the patient so it can continuously monitor respiration rate, pulse rate, blood oxygen saturation and airway patency. Moreover, locating the monitoring device 1 on the neck 180 provides a more accurate and timely measurement than conventional monitoring devices.

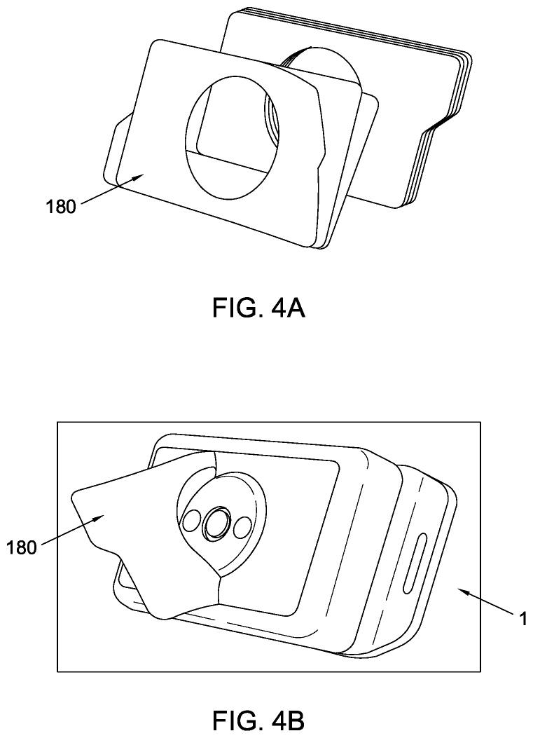

The monitoring device 1 is mounted to the neck 190 using disposable adhesive pads 180 as shown in FIGS. 4A and 4B. The pads 180 are provided with a removable protective cover 185 which is removed to reveal the adhesive surface so the monitoring device 1 can be placed on the neck 190 of the patient.

In addition to the disposable adhesive pads 180, the monitoring device can also be provided with a strap or band 165 which is non-strangulating and which attaches to the attachment points 160 located at either side of the monitoring device 1. The monitoring device 1 is typically mounted using only the adhesive pads 180 as these are more easily applied, however the strap 165 can be used to secure the monitoring device 1 if the adhesive pads 180 are insufficient. The strap 165 can also be used in situations where the patient is being transported or needs to be treated in a way that may cause the monitoring device 1 to fall off the neck 190.

The monitoring device 1 can be unmounted by pulling the adhesive pad 180 away from the neck 190 in a similar manner to a plaster. The surface of the monitoring device 1 is then cleaned, a new adhesive pad 180 is applied, and the monitoring device 1 is then ready to be re-used.

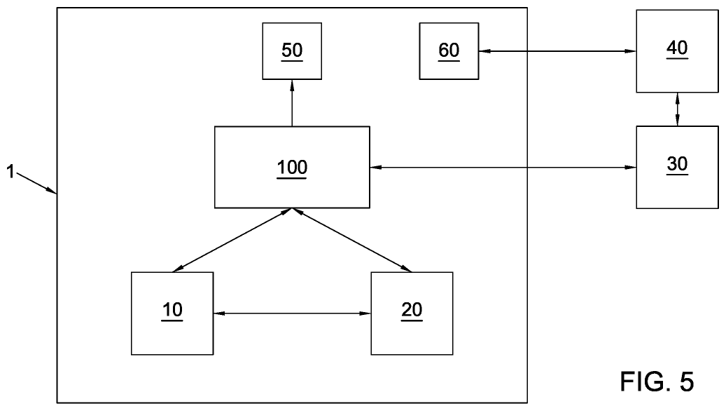

FIG. 5 shows a further schematic of the monitoring device 1 which can interact with a remote storage server 30 and/or an external device 40. As well as being transferred to the integrated storage device 50, the measurements and alerts recorded by the processor 100 are also transferred to the remote storage server 30 via a wireless network interface. Multiple monitoring devices 1 can be connected to a remote storage server 30 making this particularly useful for health care workers monitoring multiple patients simultaneously.

The monitoring device 1 further comprises a Bluetooth interface 60 which enables the device to interact with an external device 40 such as a mobile phone or computer. The external device 40 can be used to monitor the measurements from the monitoring device 1 and any alerts issued by the processor 100 can be issued on the external device 40.

The external device 40 can also control the monitoring device 1 such as by customising the pre-defined acceptable ranges for the patient's vital signs, the predetermined acceptable ranges for the rate of change of the vital signs, and the threshold value allowed for divergence of measurements of vital signs measured by both sensors.

The external device 40 can also monitor the battery-life of the monitoring device 1 and issue an alert accordingly.

The external device 40 can be connected to multiple monitoring devices 1 simultaneously. The measurements and alerts recorded by the processor may be transmitted directly to the external device 40 via the Bluetooth interface 60.

The external device 40 can also connect to the remote storage server 30 via a wireless network interface to access data recorded by the processors 100 for multiple monitoring devices 1. This allows the user to monitor the results from multiple monitoring devices 1 simultaneously.

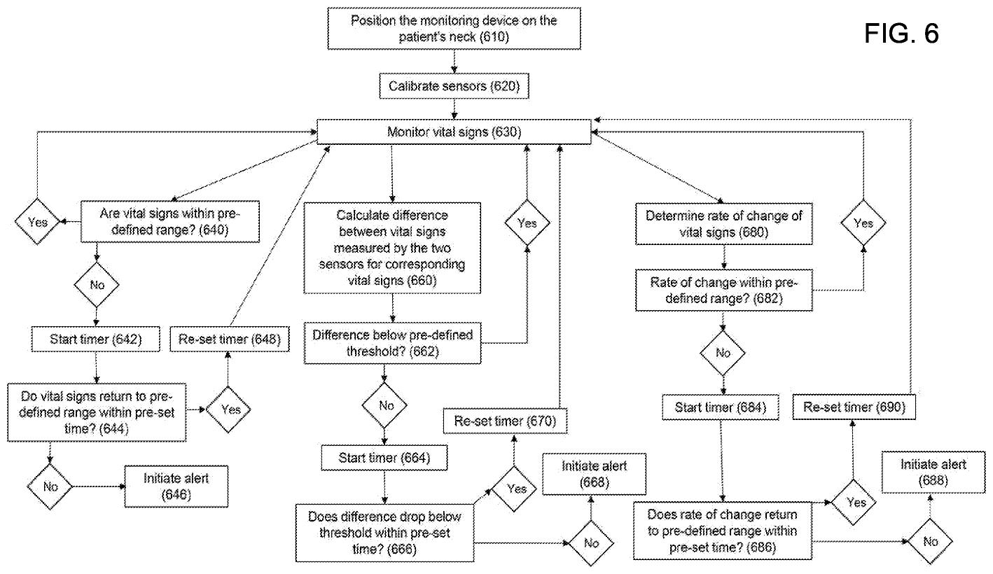

FIG. 6 shows a method of monitoring a patient's airway using the monitoring device 1. At step 610, the device is placed non-invasively on the patient's neck 190 using the adhesive pads 180 and, if necessary, the strap 165.

Once the device is activated, at step 620 it will self-calibrate (for example by comparing the sensor signals against internal or external references, and adjusting as necessary) and the processor 100 will begin processing signals from the first sensor 10 and the second sensor 20. The monitoring device 1 will provide an alert when the signals to the processor 100 are adequate and automatic monitoring of the patient's vital signs will begin. The alert will be provided by the either the audible alarm 120 or the visual indicator light 140.

The monitoring device 1 will continue to monitor the vital signs (step 630) and display the measurements 200, 300, 400, 500 on the display for the health care worker to see without the need for constant attention and manual reassessment of the patient's airway, respiration rate, pulse rate and blood oxygen saturation.

The processor 100 will continuously process the signals from the first sensor 10 and the second sensor 20. The data is stored in the integrated storage 50 and transmitted to the external device 40 and/or remote storage server 30 via the wireless network interface. The data can be monitored either by viewing the display 5 directly, or viewing the data on the external device 40 such as a computer, tablet or mobile phone. The latter is useful if multiple patients are being monitored, for example in multiple locations.

At step 640, the processor 100 will continuously monitor the vital signs and determine whether or not they are within their respective pre-defined ranges. If they are within the pre-defined ranges the processor 100 will continue monitoring the vital signs as normal.

If one or more of the measurements 200, 300, 400, 500 monitored by the first and second sensors 10, 20 are outside of the pre-defined ranges, this may indicate that the patient's airway may be blocked or that they have stopped breathing. If the one or more vital signs are outside of the pre-defined range a timer is started (step 642). In step 644, it is determined whether the vital sign returns to within the acceptable range within a pre-set time limit. If the vital sign does not return to within the acceptable range within the pre-set time limit, the processor will initiate an alert at step 646 via one or both of the audible alarm 120 or the visual indicator light 140. The choice of alert method will depend on the environment, for example in military uses an audible alert may not be preferable and so can be deactivated. The alert may also be issued on the external device 40. If the vital sign returns to within the acceptable range within the pre-set time limit, the timer is reset to zero (step 648).

If the blood oxygen saturation goes outside 90% to 100%, and does not return to this range within 15 seconds, an alert is issued. If the pulse rate goes outside of 50 to 120 beats per minute and does not return to this acceptable range within 15 seconds, an alert is issued. If the respiration rate goes outside of the range of 8 to 27 breaths per minute, and does not return to this range within 15 seconds, an alert is issued.

At step 660, the processor 100 will also continuously monitor whether there is any discrepancy between the values measured by the first sensor 10 and the second sensor 20 for the same vital sign (i.e. the pulse rate measured by the first sensor 10 and the second sensor 20 can be compared, and the respiration rate measured by the first sensor 10 and the second sensor 20 can be compared). In normal operation, the values for respiration rate and pulse rate measured by the two sensors should be the same (or within a certain predefined range of each other) indicating that both sensors are working correctly.

If the values for respiration rate and pulse rate recorded by the first and second sensor 10, 20 are different, this may indicate an error associated with one of the sensors. At step 662, the processor 100 will determine if the differences between the measurements from each of the first and second sensor (for pulse rate and respiration rate) are below a pre-defined threshold value. If it is then the monitoring device is allowed to continue operating. The pre-defined threshold can be pre-programmed into the device, or it can be customised using the external device 40 via the Bluetooth interface 60 (by a user with a high level of authorisation, not necessarily possessed by the health care worker).

If the difference between the measurements from the first and second sensor 10, 20 exceeds the pre-defined threshold a timer is started (step 664), and it is determined whether or not the divergence between the vital signs recorded by the first sensor 10 and the second sensor 20 drop backs to below the threshold value within a pre-set time limit (step 666). If the divergence between the vital signs recorded by the first sensor 10 and the second sensor 20 does not drop back to below the threshold value within a pre-set time limit, then at step 668 an alert is issued using either the audible alarm 120 or the visual indicator light 140. This will notify the nearby care worker that the monitoring device 1 is malfunctioning so it can be replaced. If the divergence drop backs to below the threshold value within the pre-set time limit, the timer is reset to zero (step 670).

The acceptable divergence for respiratory rate is 3 breaths per minute. If the values measured by the first sensor 10 and the second sensor 20 diverge by more than this, a timer is started. If the divergence does not fall back below the threshold value within 1 minute, an alert is issued.

The acceptable divergence for pulse rate is 12 beats per minute. If the values measured by the first sensor 10 and the second sensor 20 diverge by more than this, a timer is started. If the divergence does not fall back below the threshold value within 30 seconds, an alert is issued.

The method further comprises step 680, in which the rate of change with respect to time of the one or more vital signs recorded by the sensors are monitored. The one or more vital signs should remain at an approximately constant level and the rate of change for each of them should be within respective pre-determined ranges.

The processor 100 will determine at step 682 whether the rates of change for the vital signs are within their respective pre-determined acceptable ranges (including positive and negative values of the rate of change) or exceeds a predetermined limit. If the rate of the change of the one or more vital signs is outside of the pre-determined acceptable range or exceeds a predetermined limit, a timer is started (step 684). In step 686 it is determined whether the rate of the change of the one or more vital signs returns to the pre-determined acceptable range/limit within a respective pre-set period of time. If not, an alert is issued at step 688. Otherwise, the timer is reset to zero (step 690).

If a patient's respiratory rate changes by more than ±30% (and remains outside of this range for a 10 minute period), an alert is issued.

If a patient's pulse rate changes by more than ±40% (and remains outside of this range for a 10 minute period), an alert is issued.

If a patient's blood oxygen decreases by more than 5% (and remains outside of this limit for a 10 minute period), an alert is issued.

The method also comprises detecting decreasing, or loss of, airway patency. On detection of one or more of snoring, wheezing or rattling, a timer is started, and an alert may be issued if the snoring, wheezing or rattling continues for a predetermined time (5 to 10 minutes). If the snoring, wheezing or rattling ceases within the predetermined time (with breathing returning to normal) the timer is reset to zero.

If the airway is fully obstructed, the loss of airway patency is be determined by detecting that the respiration rate has dropped to zero. If the respiration rate drops to zero, a timer is started and an alert is issued after 20 seconds.

The above method using the monitoring device 1 provides a more decisive and earlier warning of respiratory failure.

The monitoring device 1 has numerous uses within the field of hospital and emergency care. During multi-casualty incidents, simultaneous triage, treatment and frequent checking of patients is required during both at the incident site and during transport. The monitoring device 1 enables the airway, respiration rate, pulse rate and blood oxygen saturation of a patient to be continuously monitored while the nearby care worker can attend to other tasks and patients. The compact nature of the monitoring device 1 means that it can be used to monitor patients as they are being transported to a hospital, or if they are being moved within a hospital, in particular if the patient has been sedated.

First responders such as paramedics are able to use the monitoring device 1 whilst attending to the numerous other tasks they will have to carry out. The attachment of the monitoring device 1 will require very little time allowing the paramedic to immediately begin other emergency tasks. Moreover, the paramedic will not constantly need to manually reassess the patient's airway as an alert will be triggered by the monitoring device 1 if necessary.

During states of emergency, hospitals may become overfilled and the need for temporary field hospitals may arise which do not have access to the same level of equipment as permanent hospitals. The monitoring device 1 can be used in this situation to monitor large numbers of patients simultaneously relatively easily.

The monitoring device 1 also has uses for home monitoring as a professional care worker is not required.

1. A monitoring device configured to be removably attached to a patient's neck, the monitoring device comprising:
an optical unit for monitoring one or more vital signs; and
an acoustic unit for monitoring one or more vital signs.

2. The monitoring device according to claim 1, wherein the monitoring device is configured to determine whether measurements of the one or more vital signs fall inside or outside of respective pre-defined ranges for the one or more vital signs.

3. The monitoring device according to claim 1, wherein the monitoring device is configured to monitor the rate of change of the measurements of the one or more vital signs, and is configured to determine whether the rate of change of the measurements of the one or more vital signs fall inside or outside of respective pre-defined ranges for rate of change of the measurements of the one or more vital signs.

4. The monitoring device according to claim 2, wherein the monitoring device is configured to issue an alert if the one or more vital signs is outside the respective pre-defined range for a time period exceeding a respective pre-set time limit.

5. The monitoring device according to claim 3, wherein the monitoring device is configured to issue an alert if the rate of change of the measurements of the one or more vital signs is outside the respective pre-defined range for a time period exceeding a respective pre-set time limit.

6. The monitoring device according to claim 1, wherein the monitoring device is configured to issue an alert if the one or more vital signs measured by the optical unit and the acoustic unit diverge by more than a threshold amount for a time period exceeding a pre-set time limit.

7. The monitoring device according to claim 4, wherein the alert is an audible alert and the monitoring device comprises a speaker for outputting the audible alert.

8. The monitoring device according to claim 4, wherein the alert is a visual alert, and the monitoring device comprises a display screen and/or a visible light source for outputting the visual alert.

9. The monitoring device according to claim 4, comprising an integrated storage means configured to store a history of the alerts.

10. The monitoring device according to claim 1, wherein the monitoring device comprises a processor for analysing the one or more vital signs.

11. The monitoring device according to claim 1, wherein the monitoring device comprises a display for displaying the one or more vital signs.

12. The monitoring device according to claim 1, wherein the optical unit comprises a reflectance pulse oximeter.

13. The monitoring device according to claim 1, wherein the acoustic unit comprises a microphone or a piezoelectric sensor.

14. The monitoring device according to claim 1, wherein the vital signs monitored by the optical unit include one or more of blood oxygen saturation, pulse rate and respiration rate.

15. The monitoring device according to claim 1, wherein the vital signs monitored by the acoustic unit include one or more of respiration rate, pulse rate and airway patency.

16. The monitoring device according to claim 1, comprising an adhesive pad for removably attaching the monitoring device to the neck of the patient.

17. The monitoring device according to claim 1, comprising a strap for removably attaching the monitoring device to the neck of the patient.

18. The monitoring device according to claim 1, comprising an integrated storage means configured to store a historical record of the one or more vital signs.

19. The monitoring device according to claim 1, wherein the monitoring device further comprises a wireless interface for communication with an external device and/or a remote storage server.

20. The monitoring device according to claim 1, wherein the monitoring device is configured to transmit a history of the one or more vital signs and/or alerts to a remote storage server.

21. A method of monitoring one or more vital signs of a patient using a monitoring device, the monitoring device comprising an optical unit for monitoring one or more vital signs, and an acoustic unit for monitoring one or more vital signs,
wherein the method comprises;
removably attaching the monitoring device to a patient's neck;
monitoring the one or more vital signs using the acoustic unit and the optical unit.

22. The method according to claim 21, comprising determining whether the monitored vital signs fall inside or outside of pre-determined ranges.

23. The method according to claim 22, wherein if the one or more vital signs are outside of the pre-determined ranges for a time period exceeding a respective pre-set time limit, the method further comprises:
issuing an alert, wherein the alert is an audible alert and/or a visual alert.

24. The method according to claim 21, comprising comparing a first value for a vital sign measured by the optical sensor to a second value for the same vital sign as measured by the acoustic sensor.

25. The method according to claim 24, comprising issuing an alert if the first and second values diverge by more than a threshold amount for a time period exceeding a respective pre-set time limit.

26. The method according to claim 21, comprising monitoring the rate of change of the measurements of the one or more vital signs, determining whether the rate of change of the measurements of the one or more vital signs falls within or outside of pre-defined ranges for the rate of change of the measurements of the one or more vital signs.

27. The method according to claim 26, comprising issuing an alert if the rate of change of the measurements of the one or more vital signs is outside of the respective pre-defined range for a time period exceeding a respective pre-set time limit.

28. The method according to claim 21, comprising monitoring one or more of blood oxygen saturation, pulse rate and respiration rate using the optical sensor.

29. The method according to claim 21, comprising monitoring one or more of respiration rate, pulse rate and airway patency using the acoustic sensor.

30. (canceled)
